(19) United States
(12) Patent Application Publication
(10) Pub. No.: US 2004/0141999 A1
(43) Pub. Date: Jul. 22, 2004

(54) Antiviral and antiretroviral radioimmunomedicaments based on (alpha)-emitters and (beta)-emitters

(76) Inventor: Wolfgang Bergter, Hannover (DE)

Correspondence Address: Gudrun E. Huckett, Ph.D., Lonsstr. 53, Wuppertal, 42289, DE

(21) Appl. No.: 10/752,563

(22) Filed: Jan. 8, 2004

Related U.S. Application Data
(63) Continuation of application No. 09/720,512, filed on Feb. 5, 2001.
Application No. 09/720,512 is a 371 of international application No. PCT/DE99/01894, filed on Jun. 29, 1999.

(30) Foreign Application Priority Data
Jun. 29, 1998 (DE) 198 28 732.1

(51) Int. Cl. A61M 36/14; A61K 51/00; A61K 39/395; A61K 39/29; A61K 39/00; A61K 39/21

(52) U.S. Cl. 424/192.1; 424/1.11; 424/141.1; 424/225.1; 424/227.1; 424/228.1; 424/208.1

(57) ABSTRACT

In a method of treating viral infections and of tumors induced thereby a radioimmunoconjugate (RIC) containing an immunologically effective component and a radioactive component is provided. The immunologically effective component contains a) a receptor molecule or a fragment thereof having affinity to an epitope of the viral structural proteins expressed on the plasma membrane of infected cells, or b) a fragment of the cellular receptor molecule modified by mutagenesis, the fragment having affinity to an epitope of the viral structural proteins expressed on the plasma membrane of infected cells. The radioactive component is an alpha emitter or a beta emitter. The therapeutical agent is effective against viral infections such as HIV, HBV, HCV, HDV, HTLV, CMV, EBV, or HHV8 infections.

This application is a continuation application of application Ser. No. 09/720,512, filed Feb. 5, 2001, which is a national stage application of PCT/DE99/01894 with an international filing date of Jun. 29, 1999, not published in English under PCT Article 21(2), and now abandoned.

### BACKGROUND OF THE INVENTION

The present invention relates to pharmaceutical preparations based on radioimmunoconjugates (RICs) to be used therapeutically in the field of virology, in particular to combat and cure, respectively, infectious diseases causes by HIV-1, HIV-2, HIV-3, HTLV-1, HTLV-2, HBV, HCV, HDV, CMV, EBV and HHV8, and tumors induced thereby. The present invention also relates to pharmaceutical preparations based on radioimmunoconjugates (RICs) which are to be used therapeutically in mammals infected with animal viruses (e.g. SIV) corresponding to HIV-1, HIV-2, HIV-3, HTLV-1, HTLV-2, HBV, HCV, HDV, CMV, EBV and HHV8.

The treatment of most viral infections—in particular of the HIV, HCV, and HBV infections—have not been solved in a satisfactory manner hitherto. Below, the pertinence of these infections, the respective state of the art, e.g., the conventional therapies as well as their limitations and problems will be exemplified on the basis of the HIV and HCV infection.

Prior Art Exemplified by HIV Infections.

According to an estimation of the WHO in December 1997 worldwide already 30.6 million people were infected with the human immune deficiency virus HIV. The HIV infection results in a progressive immune defect by a loss of the CD4 receptor bearing (CD4⁺) T lymphocytes (helper cells) infected with HIV. Patients with a symptomatic HIV infection or terminal cases suffering from an HIV infection (AIDS) exhibiting a number of CD4 cells less than 350 per μl blood and/or a virus load of more than 30.000 viral RNA copies (vRNA)/μl in the serum are nowadays recommendedly treated by an antiretroviral therapy, preferably with a medical triple combination to be applied orally, the combination consisting of two inhibitors of the reverse transcriptase (RTI) and a protease inhibitor (PI) (Brodt et al. 1997). It is true that the replication of the HI viruses is disturbed and slowed down by these drugs but HIV replicating host cells cannot be eliminated. The HIV infection thus pertains forever as an infection threatening lives. A cure of the HIV infection is not yet possible. Apart from that, the conventional treatment of one HIV infected (AIDS) patient amounts to costs of approx. $7,500 and results in continuously occurring side effects of partially significant extent.

Conventional immunotherapeutical approaches for the treatment of diseases triggered by HIV or HHV8 (AIDS and Kaposi-Sarcoma, respectively, has not been a satisfying option for the treatment of such diseases until today. It is true that HIV and HIV infected cells can be labeled with radioactively non-labeled (“cold”) antibodies but these antibodies do not induce the desired immune response leading to the elimination of the infected cells because the immune response is compromised by the HIV infection anyway. The physiological cytotoxic immune response impaired due to the HIV infection must be substituted by a targeted artificial induced death of all HIV infected cells, if possible at all.

Prior Art Exemplified by HCV Infections.

Also a very great number of people, that is more than 1% of the world's population, are infected with the hepatitis C virus (HCV) (Böker and Manns 1997). The acute HCV infection does not cure spontaneously in 80% of the cases but becomes a chronic infection. Absent a therapy about 30% of the patients suffering from chronic hepatitis C develop cirrhosis of the liver including all hepatic and extra-hepatic complications within a term of 20 to 30 years: Not only portal hypertension, ascites formation, bleeding of the esophageal varices, hepatic encephalopathy and hepatocellular carcinoma occur but also cryoglobulinemia with arthralgia, pruritus, purpura, neuropathy and glomerulonephritis, cryoglobulin-independent glomerulonephritides, sicca syndrome, various autoimmune diseases and even possibly lymphomas (Maier 1997, Manns and Rambusch 1997, Petry et al. 1997). In fact, worldwide more people die from the consequences of a hepatitis C infection than from the consequences of the HIV epidemic presently. Hepatitis C, thus, constitutes a gigantic unsolved social problem.

As the causative agent of the hepatitis C, known since 1978 as non-A-non-B-hepatitis (Alter et al. 1978), the hepatitis C virus (HCV) could be identified 1989 for the first time (Choo et al. 1989). The particle of approx. 30-65 nm classified to belong to the family of the flavi viruses comprises a single-stranded RNA genome encoding a polyprotein of about 3000 amino acid in length.

This polyprotein is cleaved and processed in a complex procedure into the 3 structural proteins C, E1, and E2 as well as the functional proteins NS1 to NS5 (Shimotohno et al. 1995). The maturation of the virions occurs in the lumen of the endoplasmatic reticulum and Golgi-apparatus. From these places the virions are transported to the cell surface where they bud out of the plasma membrane (Pozzetto et al. 1996).

By now not much is known as regards the integration of viral or virus induced proteins into the cell membrane of HCV infected cells (Selby et al. 1993). One may assume, however, that structural proteins such as E1 and E2, conceivably even other viral and virus induced proteins, are formed on the surface of HIV infected cells.

The viral RNA is protected by a nucleocapsid of core proteins (C) and enveloped by a lipid envelope including the surface proteins (E1 and E2), integrated into the envelope (Zilpert and Roggendorf 1997). Supported by these surface molecules the adsorption of the virus occurs, presumably via an epitope that is partially independent on the hyper-variable regents (Rosa et al. 1996), in a targeted manner to a receptor of the host cell, conceivably to the LDL receptor (Seipp et al. 1997).

The adsorption can be inhibited by antibodies in the serum of HCV infected persons. The so called neutralizing epitopes are subject to a high mutation activity of the virus and are therefore variable such that an effective protection against all isolates of a quasi species cannot be established (Farci et al. 1996). This pronounced variability presently drastically complicates the development of a HCV vaccine (Pozzetto et al. 1996).

At present, medicine has not yet been capable to accomplish a, strictly speaking, curing, that is, a complete elimination of the HC-viruses inducing the disease also in regard of treating hepatitis C patients. Only one standard therapy for chronic hepatitis C has been established in the past years, that is the treatment with Interferon-alpha (IFN-α).

IFN-α is a physiologic protein to the cytokines and produced by monocytes and activated B-lymphocytes in response to a virus infection. The rationale of the HCV therapy are the properties of IFN-α inhibiting replication and modulating the immune response. On the one hand, this cytokine is able to inhibit the viral replication cycle. On the other hand it is able to activate cytotoxic T cells which, in turn, selectively kill HCV infected cells.

At present modes of administration with 3×6 million units IFN-α/week as a subcutaneous injection during a period of 3-4 months and, if the patient reacts, for further 8-9 months are recommended (Böker and Manns 1997).

Only about 20% of the people chronically infected with HCV and treated with IFN-α permanently respond to the therapy (Zilbert and Roggendorf 1997). 80% of the patients remain infected and have to expect prominent complications of the disease. A particularly bad responder to a IFN-α therapy is the HCV genotype 1b dominant in our western countries. The reason for this is not known until hitherto.

The costs for the treatment for each IFN-α therapy approach interrupted for lack of success after 3 months (>50% of the cases) amount to about $2,500 presently, and to about $10,000 in all other cases, including additional 25% of therapy failures, the so called relapsers. A successful IFN-α therapy thus costs more than $25,000 (calculated according to Berg and Hopf 1997).

A broad range of side effects may be elicited by IFN-α (Berg and Hopf 1997). Almost always the patients suffer, at least initially, from partially severe-influenza-like discomforts, which cause some patients to even abandon therapy. Sometimes, even changes of the blood count, autoimmune diseases and neurologic and psychic diseases are induced that require a discontinuation of treatment to be arranged by the therapist.

As an alternative to the IFN-α monotherapy the combination treatment with IFN-α and ribavirin is presently in the process of being established, which latter treatment exhibits an increased response-rate according to reports of other groups (e.g. Schvarcz et al 1995) and own experiences (Fetzer et al. 1997). The basic problem of this therapy strategy remains also, however, that only a slight inhibition of the viral replication and no elimination of the viral genome can be accomplished with a nucleoside analogue such as ribavirin (Reichard et al. 1993).

In the meantime it has been shown that an initially successfully treated hepatitis C may flare up even after years (Vento et al. 1996). This means that the HCV infection, in spite of the use of IFN-α and combination therapy with ribavirin, is a hitherto in most cases incurable disease.

An effective vaccine is not to be expected in due course. HCV is characterized, as is the human immunity deficiency virus HIV by a strong mutagenicity in the regent of its surface proteins E1 and E2, in particular in the region of the neutralizing epitopes. This enables the virus to escape again and again the attacks of the immune system. The spontaneous or therapeutically accomplished elimination of a HCV-isolate does not coincide with an effective immunological protection against an infection with another HIV isolate (Purcell 1997).

The clinical test of a potential vaccine is additionally difficult and time-consuming. Firstly, there is only one animal model available which is difficult to handle, however. This animal model is that of chimpanzees infected with HCV. Secondly, the test in humans is not without risk and will require years before reliable data in regard to the efficiency of the vaccination can be made. Thirdly, even an HCV vaccine introduced to the market already today could not change the fact that worldwide an estimated 40 million people remain HCV infected and require an effective antiviral therapy.

Since molecular biological methods for the targeted elimination of the HIV and HCV genome in infected cells will not be available within a foreseeable time frame, the therapy of the HIV and HCV infection must aim at eliminating the HIV and HCV replicating cells with presently feasible therapeutic methods.

Background of the Radioimmunotherapy in Virus Infections.

The infections mentioned have in common that their course is chronic and is associated with a high morbidity and mortality. The virus replication can be suppressed more or less well in the meantime by means of antiviral and antiretroviral, respectively, pharmacons. A curing of the hepatitis B and C is presently accomplished only in a limited portion of the cases, however, and an HIV infection presumably can be eliminated not at all by inhibiting the virus replication alone.

Object of an effective antiviral and antiretroviral, respectively, therapy must therefore always be the infected cell per se.

The treatment with radioisotopes is known in Germany for more than 50 years as radio iodine therapy from the treatment of benign and malignant diseases of the thyroid gland, and it is known to be the most important therapeutic method of the nuclear medicinal therapy which method has proven to cause only few side effects. Late complications, in particular the induction of a malignant tumor could not be detected until today (Moser 1996).

More recent options for therapies with radioisotopes have in the meantime arisen with ¹³¹I-labeled meta-iodo-benzyl-guanidine (MIBG) in the treatment of metastasized phaeochromocytomas and neuroblastomas, by the radiosynoviorthesis for the rheumatoid arthritis, by intracavitary instillation of ⁹⁰Y-silicate in the pleura- or peritoneal carcinomatosis, the palliative pain therapy of metastasis of the skeleton with substances of ⁸⁹Sr having an affinity to bones, or the radiophosphorus treatment of the polycythaemia vera (Moser 1996). Presently a therapy with ¹³¹I-labeled anti-CEA IgG for the colorectal carcinoma (Blumenthal et al. 1992, Blumenthal 1994) and the B cell lymphoma (Kaminski et al. 1993, Press et al. 1993, Press et al. 1995, Press et al. 1995) are in clinical trials.

There does not exist any experience regarding the use of radioisotopes for the therapy of the HIV infection. Whereas gamma radiation in small dosage evidently can induce an enhanced HIV expression (Xu et al. 1996), it was shown in animal experiments using the SIV infection of a macaque that a carefully directed radiation of lymph nodes may lead to the reduction of the virus load in the peripheral blood and to a standstill of the progression of the disease (Fultz et al. 1995). A radiation of the entire body of HIV infected patients, however, is only unspecifically effective and requires a high dose of radiation. The effect is thus principally uncertain and the price due to undesired radiation damages is high.

Such targeted elimination of virus infected cells is accomplishable with radioimmunopharmacons according to the invention, which pharmacons comprise an α- or β-emitter as a radioactive component. The range of the β-radiation is about several mm. The problem thus resides in that it cannot be excluded that surrounding non-infected cells are damaged. Therefore, it would be generally more favorable to employ α-radiation effecting a higher linear energy transfer and having a smaller range.

### SUMMARY OF THE INVENTION

The problems described are solved according to the present invention in that pharmaceutical preparations are provided containing immunological effective molecules such as monoclonal antibodies (mAb) or cellular receptors for the virus binding conjugated to an α- or β-emitter. Such construct will be termed radioimmunoconjugate (RIC) hereinafter. According to the invention even small peptides, for instance designed and constructed by means of molecule design, are suitable as long as they bind to viral antigens or cellular antigens specific for the infected cell, as do antibodies. The smaller the molecular weight of such peptides, the easier they pass the blood-brain-barrier such that infected cells in the central nervous system can be reliably reached and eliminated as well.

A further aspect of the present invention relates to the use of such RICs for the preparation of a medicament for the treatment of viral infections and tumors induced thereby. According to a preferred embodiment the infection to be treated is an HIV-1, HIV-2, HIV-3, HTLV-1, HTLV-2, HBV, HCV, HDV, CMV, EBV, or HHV8 infection and a tumor in a human induced by such infection and the corresponding disease respectively (infection with a virus corresponding to the above mentioned viruses and tumors induced thereby), in a mammal such as monkey or mouse.

A further aspect of the present invention relates to a kit comprising a RIC according to the invention, preferably in lyophilized form, a column for chromatographic separation methods and, optionally, a solvent for the antibody/receptor and its fragment, respectively, optionally an oxidizing agent such as chloramine T or a chelating agent to perform the conjugation of the radionuclide to the molecule specifically recognizing the infected cell.

Compounds of the following general formula may be employed as RICs for the pharmaceutical preparation according to the present invention:

a) binding molecule—alpha emitter;

b) binding molecule—beta emitter.

Preferred are in particular compounds of the formula:

a1) mAb—alpha emitter;

a2) mAb fragment—alpha emitter;

a3) virus receptor or virus receptor fragment—alpha emitter;

a4) synthetic peptide (molecular design)—alpha emitter.

The specificity of the mAb shall be particularly directed to epitopes integrated into the plasma membrane of the cells. These are

a) epitopes of the surface or transmembrane glycoproteins of HIV-1, 2, or 3, e.g. gp120 and gp41 of HIV-1 and of the corresponding structural proteins of HIV-2, HIV-3;

b) epitopes of the surface glycoproteins of HBV, e.g. HBsAg (SHBs, MHBs, LHBs);

c) epitopes of the surface glycoproteins of HCV, e.g. E1 and E2;

d) epitopes of the surface of HHV8;

e) epitopes of the surface or transmembrane glycoproteins of other retroviruses such as gp46 and gp21 of HTLV-1 and HTLV-2, respectively;

f) peptide T20/DP178 (Kilby et al. 1998, Su et al. 1999) or a fragment thereof corresponding to a portion at the C-terminus of gp41;

g) epitopes of the gp220/350 complexes of EBV (Darai et al.).

In the alternative (monoclonal) antibodies specific for virus induced proteins and peptides integrated into the plasma membrane of infected cells (e.g. protein/peptides of the MHC).

As monoclonal antibodies the following may be employed:

a) murine monoclonal antibodies;

b) human and humanized monoclonal antibodies;

c) antigen binding fragments (Fab, Fab′, or F(ab)₂) of murine or human or humanized monoclonal antibodies.

Various molecules could be identified as virus receptors. Such molecules are therefore to be employed, inter alia:

a) CD4 receptors (in case of HIV infections);

b) LDL receptors (in case of HCV infections);

c) ASGPR (asialoglycoprotein receptor; Treichel et al. 1997) and other receptors (in case of HBV infections).

Further receptors as specifically required by the respective virus to invade and infect the cells may be employed depending on the infection to be treated. Optionally, suitable antigen binding fragments (peptides) of the binding proteins exhibiting improved pharmacodynamics and pharmacokinetics properties may be prepared and the respective binding molecule (or a fragment thereof) may be modified by mutagenesis such that improved binding properties for the radioimmunopharmacon result.

Accordingly, particularly preferred compounds according to the above formula a) and b) are

a5) gp41 (of HIV-1)—alpha emitter;

a6) gp120 (of HIV-1)—alpha emitter;

a7) CD4 receptor—alpha emitter;

a8) E1 (of HCV)—alpha emitter;

a9) E2 (of HCV)—alpha emitter;

a10) small peptide according to molecular design—alpha emitter.

Suitable radionucleotides are ¹³¹I, ³²P, ⁹⁰Y, ⁸⁹Sr, ¹⁷⁷Lu, ¹⁸⁶Re, ¹⁸⁸Re, ¹⁰⁵Rh, ⁹⁷Ru, ⁴⁷Sc, ¹⁵³Sm, and ¹⁴⁹Tb (beta emitter) and ²¹²Bi, ²²⁵Ac, ²¹³Bi, and ²²³Ra (alpha emitter).

A therapeutic option (in case of HIV infection) resides in the coupling of a radioisotope to a HIV specific antibody recognizing an epitope of a viral structural protein presented on the cell surface of HIV infected cells, and binding thereto. Preferably a combination of a human gp41 specific monoclonal antibody with any of the following isotopes may be employed: ¹³¹I, ³²P, ⁹⁰Y, ⁸⁹Sr, ¹⁷⁷Lu, ¹⁸⁶Re, ¹⁸⁸Re, ¹⁰⁵Rh, ⁹⁷ Ru, ⁴⁷Sc, ¹⁵³Sm, and ¹⁴⁹Tb (beta emitter) and ²¹²Bi, ²²⁵Ac, ²¹³Bi, and ²²³Ra (alpha emitter).

The retroviral transmembrane glycoprotein gp41 is firmly integrated into the viral and cellular lipid membrane and, thus, in contrast to the surface protein gp120, cannot dissociate from the virus or the HIV infected cell by shedding. Particularly advantageous are antibodies specific for a strongly conserved epitope, therein human antibodies are preferred.

Particularly suitable as component of a radioimmunoconjugate and amply characterized is, as an example, the human monoclonal antibody 2F5 that binds to a strongly conserved and therefore widely spread epitope among the various HIV-1 isolates (Muster et al. 1993).

Another option (in the case of HIV infection) resides in the corresponding conjugation of CD4 molecules and fragments, respectively, of CD4 molecules with radioisotopes. Both α- and β-emitter such as ¹³¹I, ³²P, ⁹⁰Y, ⁸⁹Sr, ¹⁷⁷Lu, ¹⁸⁶Re, ¹⁸⁸Re, ¹⁰⁵Rh, ⁹⁷Ru, ⁴⁷Sc, ¹⁵³Sm, and ¹⁴⁹Tb (beta emitter) and ²¹²Bi, ²²⁵Ac, ²¹³Bi, and ²²³Ra (alpha emitter) may be employed. The conjugates may be used effectively in order to accomplish a curing of the infection with HIV-1 or HIV-2 by targeted killing of all infected cells.

CD4 molecules are integrated into the cell membrane of T lymphocytes, have a molecular weight of about 55.000 Dalton and play a key role in the defense of infections by means of their receptor moiety directed to the exterior (Gaubin et al. 1996). Additionally, they play a major role also for the binding of HIV to these cells (Dalgeish et al.) HI viruses use the CD4 receptor as the site on the exterior of the cell membrane to couple to the cell. The exact site where the viral gp120 surface glycoprotein binds could be localized as the amino terminus of the CD4 molecule. It is termed as V1 domain (Richardson et al. 1988, Arthos et al. 1989).

Based on this knowledge a number of investigations have already been conducted, which investigations were to clarify whether synthetic CD4 molecules or portions of the protein (peptide) are therapeutically applicable. It could be shown that such molecules cover the CD4 binding site of the viral surface glycoprotein gp120 and neutralize HI viruses (Deen et al. 1988, Byrn et al. 1989, Clapham et al. 1989, Watanabe et al. 1989). Thus, uninfected cells may be protected from an infection with HIV. For the therapy of an existing HIV infection the application of such proteins or peptides alone will not be sufficient, however, because the HIV genome in the infected cells is not destroyed and new HI viruses are continued to be replicated unhindered.

A further option (in case of HIV and HCV infections) are conjugates of a monoclonal antibody or its antigen binding fragment and the receptor molecule of HIV/HCV or a fragment thereof, respectively, and an α- or β-emitter.

The preparation of murine, humanized and human monoclonal antibody is known (reviews: Lidell and Weeks 1995, Peters et al. 1996). The isolation of monoclonal antibodies specific for SIV and HIV is one basis of the HIV research (Bergter 1990). The generation and conjugation of radioactive isotopes is also sufficiently described (review in: Eckert and Kartenbeck 1996).

Alpha radiation is a corpuscular radiation. By emission of alpha particles the nucleus of an alpha emitter loses a positive charge of +2e and a mass of about 4 amu. The emitted alpha particles have a range of less than 100 mm which is significantly lower than the range of electrons of beta emitters. However, they exhibit a higher energy and effect a remarkably denser ionization than do beta emitters (Zalutzky and Bigner 1996). This results in a more intensive damaging of the plasma membranes of infected cells and in a significantly increased cytotoxicity whereas non-infected cells are exposed to a smaller unspecific radiation effect. This is important in the event that, for example, HIV infected cells distribute disseminatedly in the entire body of the patient.

Most of the 100 radionuclides emitting alpha particles are unsuitable for a radioimmunotherapy due to their long half life. Many of them are difficult and in a quantity to low to prepare such that only few alpha emitters have been considered hitherto for a clinical trial. Most experience is available with the application of astatine-211 (²¹¹At) and bismuth-212 (²¹²Bi) (Zweit 1996).

Astatine-211 is particularly suitable for the therapeutic purposes according to the present invention because it decomposes via two routes, both resulting in the emission of alpha particles as it emits X-rays exhibiting attentive sufficiently high to investigate blood and tissue samples with a gamma counter or to analyze the therapeutic treatment with imaging methods such as the single photon emission computer tomography (SPECT) (Turkington et al. 1993) and because it has a half life of only 7 h.

Likewise advantages within the meaning of the present invention is bismuth-212 with a half life of 70 min that had previously been conjugated with monoclonal antibodies and successfully used in an animal model for the elimination of malignant cells (Kozak et al. 1986, Macklis et al. 1988).

Another suitable alpha emitter having a half life of 46 min is bismuth-213 (²¹³Bi), that may be generated from the parent nuclide actinium (²²⁵Ac) by means of generator.

Generally speaking, all alpha and beta emitters are particularly suitable which have a half life of 10 d or less as the patients will then be exposed to a remarkable dose of radioactivity only for a relatively short time period.

The effects of radioactive radiation on cells and genetic material (DNA) have been described sufficiently (reviews in: Kauffmann et al. 1996, Sauer 1998). Basis of the radiobiological effects is the structural damage of the genetic material (DNA) of virus infected cells (in case of the HCV infection of the hepatocellular DNA). DNA thus modified encodes defective proteins which, in turn, lose their function. The same applies to enzymes that are indispensable for the virus replication which enzymes lose their function thereby. In addition, metabolic changes in the virus infected cell occur. This results in the elimination of virus infected cells by reproductive cell death or by apoptosis. Additionally, viruses may be inactivated directly as well by means of ionizing radiation (Follea et al. 1996).

Under oncological aspects radiation is successfully used for the therapy of the AIDS associated Kaposi sarcoma induced by the human herpes virus 8 (HHV 8) (Conill et al. 1997).

Experience regarding the effect of ionizing radiation on the replication of HIV are hardly available. Whereas gamma radiation in small dose evidently can induce an enhanced HIV expression (Faure et al. 1995, Xu et al. 1996), so far an acceleration of the HIV replication due to a therapeutic radiation of HIV associated tumors was not detected (Lotz et al. 1990, Plettenberg et al. 1991, Scheidegger et al. 1991, Stanley et al. 1991, Krain and Dieckmann 1994, Saran et al. 1995, Swift 1996, Saran et al. 1997). Rather, it was shown in animal experiments using the SIV infection of a macaque that a carefully directed radiation of lymph nodes may lead to the reduction of the virus load in the peripheral blood and to a standstill of the progression of the disease (Fultz et al. 1995). A radiation of the entire body of HIV infected patients, however, is only unspecifically effective and requires a high dose of radiation. The effect is thus principally uncertain and the price due to undesired radiation damages is high.

The targeted employment of free radioisotopes for the treatment of viral (HIV-1, HTLV-1, HBV, HHV8, HIV-2, HIV-3, HTLV-2, HCV, HDV, CMV, EBV etc.) infections have not yet been tested as yet. Their employment is legitimate, however, in the therapy of infectious diseases that are characterized by their chronic cause, their frequency and severity of their secondary symptoms, and an inefficient conventional therapy.

Basis of a radioimmunotherapy of the above mentioned viral infections with the immunoglobulin conjugates according to the present invention is that viral antigens or virus induced antigens are integrated into the outer membrane of infected cells. This is the property that has been proven in regard of many viruses, e.g., for the flavi viruses (Westaway and Goodman 1987, Ng et al. 1992), HSV (Schlehofer et al. 1979), influenza viruses (Boulan et al. 1980. Ciampor et al. 1981, Kohama et al. 1981, Hughey et al. 1992), rabies viruses (Revilla-Monsalve et al. 1985), EBV (Liebowitz et al. 1986), HBV (Saito et al. 1992, Gerber et al. 1988, Chu et al. 1997) and HIV (Timar et al. 1986, Rusche et al. 1987, Feremans et al. 1988, Desportes et al. 1989, Ikuta et al. 1989, Dennin et al. 1991, Dudhane et al. 1996).

For the radioimmunopharmacons and RICs, respectively, of the present invention which are based on immunoglobulins monoclonal antibodies and their antigen binding fragments, each being capable to recognize and bind an epitope of the viral and virus-induced antigens on the plasma membrane of infected cells should therefore be employed as immunologically effective components of the radioimmunoconjugates. Particularly suitable are monoclonal antibodies against an epitope of a viral antigen as strongly conserved as possible. The alpha or beta emitter conjugated to the monoclonal antibody or its antigen binding fragment specifically damages the virus replicating cell. In case of treating the HCV infection antibodies against an epitope on the proteins E1 or E2 as strongly conserved as possible, or against the transmembrane protein of HCV (Santolini et al. 1995) included by the NS2 region are preferred.

A particularly preferred embodiment of the present invention is directed to the simultaneous application of “cold”, e.g., non-radioactively labeled monoclonal antibodies and receptors or their fragments. The administration of the cold antibody/receptor/fragments may happen prior to (pretargeting), after, or simultaneous with the radioimmunoconjugates according to the present invention. Thereby, free virus particles can be captured and neutralized. The cold antibody may be identical with the “hot” antibody. Alternatively, it may also be a different antibody. It is only essential that the cold antibody binds to the infected cell.

Background of a radioimmunotherapy of viral infections with cell receptor conjugates is not only the expression of viral proteins on the surface of infected cells but also that the adsorption of the viruses to the host cells is mediated by a receptor on the cell membrane in a targeted manner. Such receptor-mediated adsorption had been confirmed already for various viruses, for example for HIV (Dalgleish et al. 1984), EBV (Fingeroth et al. 1984), polio viruses (Leon-Monzon et al. 1995), rabies viruses (Lentz et al. 1986), influenza C viruses (Nishimura et al. 1988), measles viruses (Dorig et al. 1993) HAV (Kaplan et al. 1996), HSV (Whitbeck et al. 1997), HBV (Treichel et al. 1997), HCV (Seipp et al. 1997), Coxsackie B3 (Shafren et al. 1997), and adeno viruses (Bergelson et al. 1998), to mention a few. Thus, HIV infected patients may be treated with, e.g., radioimmunoconjugate (a7). Likewise, the treatment of hepatitis C patients may occur with a conjugate consisting of LDL receptor (or a fragment thereof) and radionuclide, preferably α-emitter, particularly preferred bismuth-213 or astatine-211, as the HCV could arrive in the cell via the LDL receptor as cellular receptor (Seipp et al. 1997).

Corresponding recombinant receptor molecules bind to viral antigens expressed on the surface of infected cells and couple, as described above, radioisotopes to the infected cell.

The successes of the therapy according to the present invention obtained with the above described radioimmunoconjugate may even be improved by a previous or concomitant decrease of the virus load by a treatment of the patient. In this regard, the modern anti-retroviral standard triple therapy for HIV infections and the IFN-α-mono or combination therapy with ribavirin in HIV and HCV infections are suitable. By means of the triple therapy, a higher dosage of the anti-HIV radioimmunopharmacon may be transported to the virus infected cells in order to eliminate them. Otherwise the RIC would be captured by free virus particles and the cytotoxic effect reduced. The IFN-α-mono or combination therapy with ribavirin causes the stabilization of the hepatocyte cell membrane, which impedes the virus particles to bud.

Accordingly, a pharmaceutical combination product is preferred that contains not only the RICs but also IFN-α and/or ribavirin and/or a protease inhibitor (PI) and/or an anti-retroviral nucleoside analogue such as AZT that is used in standard therapies in case of HIV infections. Likewise preferred are those products that additionally contain unlabeled antibodies, receptors or fragments thereof according to the definition in any of claims 1 a) to c).

A further preferred variant for the treatment of infected patients with the RICs according to the present invention resides in that (still) uninfected cells are protected from virus in that the respective receptors (in case of HIV the CD4 receptor) are blocked with unlabeled molecules. Particular suitable for this purpose are anti-CD4 receptor antibodies and those fragments of the antibodies exhibiting a sufficiently high affinity for the receptor. Synthetic peptides having a receptor affinity are suitable as well and may be applied prior to, simultaneous with, or even after the administration of the RICs. A previous and simultaneous administration is preferred.

The radioimmunoconjugate according to the present invention is applied parenterally, preferably intravenously. Usually the stationary accommodation for several days (2 to 10 days, generally 3 to 7 or 8 days) is required in order to shield the patient from the surrounding until the radiation eased off.

The RIC specifically binds (by means of monoclonal antibodies, their fragments and by means of receptors and their fragments, respectively) to the respective epitope of a retroviral transmembrane or surface glycoprotein (or of the receptor) integrated into the cell wall of the virus replicating cell. The radioisotope thus fixed to this cell emits radiation into the closer surroundings with damages particularly the virus infected cell occupied with various radioimmunoconjugates.

After administration of the radioimmunopharmacon (including the administration to HIV patients) one has to expect transiently in particular hematological and hepatic side effects and an increased risk for opportunistic infections because one has to expect that, apart from a general myelotoxicity, the number of the T4 helper cells will decrease significantly. Since this is, however, mainly due to a loss of HIV infected T4 helper cells which are disordered in their normal function, such loss is a declared object of the therapy. For this reason one may optionally combine the radioimmunotherapy with a stem cell transplantation. As T4 precursor cells are not infected with HIV as they are lacking CD4 receptors, the regeneration of the T4 helper cell population will occur. This phenomenon is likewise observed in the pre-described investigation performed on animals in experiments (Fultz et al. 1995).

A preferred embodiment of the invention relates to the conjugate a7) and its use for the treatment of HIV infections. CD4 molecules may be recovered from tissue cultures on a large scale (Deen et al. 1988, Glick and Pasternak 1995). The purification of the CD4 molecules is possible by means of established molecular biological purification methods. They may, for example, be isolated directly from the cell membrane by differential extraction by means of non-ionic detergents (Eckert and Kartenbeck 1997 ¹) by purification from tissue culture supernatants such as described in Deen at al. 1988, by a biomagnetic separation (Deutsche Dynal GmbH, Hamburg), or by other methods. The preparation of conjugates of proteins and radioactive isotopes may occur, to mention an example, with the chloramine T method (Hunter and Greenwood 1962, Eckert und Kartenbeck 1997 ²).

Another form of this embodiment relates to the use of synthetic CD4 fragments exhibiting an affinity for gp120. Such fragments may be produced on a large scale in a prokaryotic host such as Escherichia coli or in a eukaryotic host such as Saccharomyces cerevisiae (Wilcox and Studnicka 1988, Martin and Scheinbach 1989).

Finally, the CD4 fragments may additionally be modified by mutagenesis (Jones et al. 1990) and be optimized for the therapeutic application regarding their pharmacokinetics, their affinity for gp120 and their capacity to pass the blood-brain-barrier. Prerequisite for the targeted mutagenesis is the knowledge concerning the role of the amino acids in the functional peptide. This knowledge may be obtained by genetic analysis, x-ray structural analysis of the three-dimensional peptide structure and may be simulated and analyzed by means of computer-supported investigations. A particularly preferred conjugate a7) consists of CD4 receptor, and any of the following radionuclides: Bi-212, Bi-213, and At-211.

The most important advantage of a therapy with radioimmunoconjugates according to the present invention as compared to the respective standard therapy (HCV: IFN-α therapy; HIV: conventional anti-retroviral therapy) is that virus infected cells are directly damaged and eliminated. This drastically increases the chance of a complete curing. Likewise, this applies to the method of liver transplantation which method is disadvantageous due to the problem of HCV recurrence (recidivism) in cases of cirrhosis of liver caused by HCV.

An advantage of the radioimmunotherapy of the present invention, in particular when employing conjugate a7), is that the HIV infected cells are selectively damaged. The radioimmunoconjugates specifically bind, by means of their protein and peptide moieties, respectively, to the (in case of the HIV infection) CD4 specific epitope of the retroviral gp120 surface glycoproteins which are exposed on the outer cytoplasmic membrane of HIV replicating cells for virus budding and transfer the radiation directly to the cell.

A further advantage of these conjugates a7) is that they are suitable for the treatment of a broad range of virus strains (HIV-1, HIV-2, HIV-3, HTLV-1, HTLV-2, etc.). This is extremely important in view of the high mutation rate of these viruses and their capacity as a result thereof to develop resistances to conventional therapeutic agents (the gp120 epitopes relevant for CD4 receptor binding are very similar in all HIV-1 and HIV-2 isolates (Sattentau et el. 1988) and essential for infectiousness).

Still a further advantage of synthetic and optionally further modified peptides exhibiting a size as small as possible but retaining their antigen specificity is that they can easily pass the blood-brain-barrier. Such conjugates are therefore suitable also for the elimination of infected cells of the central nervous system.

The crucial fact is that a CD4 molecule or a part thereof is coupled to a radionuclide in order to treat the HIV infection and to accomplish the object to eliminate the virus infected cells. The benefit of the nuclear medical therapy of HIV infections as described results in that this virus infection is, compared with other viral infections, a dangerous chronic and presumably in almost all cases mortal infection with lenti viruses that is incurable with the conventional pharmaceuticals. As no reliable vaccine against the HIV infection will be available in the near future (Gallo, Reuters News, May 1997), and, as meanwhile HIV strains have developed which are one-fold and several-fold resistant against conventional anti-retroviral agents (Erickson and Burt 1996, Imrie et al. 1997), novel therapeutic strategies as that described above are of great importance.

The methods of generating CD4 molecules, to develop CD4 fragments exhibiting gp120-affinity by DNA recombinant techniques and the methods to label solvable proteins in vitro with radioisotopes belong to the state of the art. The conjugation of radioisotopes to CD4 molecules or corresponding CD4 fragments will be illustrated in the examples.

Finally, a great advantage is the good subjective tolerance and the objectively few side effects of radioimmunological therapies compared with the conventional anti-retroviral therapy. It is true that the radiation energy of an α- or β-emitter such as ¹³¹I has a radiation range of 1 up to 40 cell diameters. Malignant transformations of healthy cells as a consequence of a damage by radiation are extremely rare, however, and can hardly be registered statistically. If a healthy cell is damaged it will in most cases likewise lose its capacity to divide. Accordingly, the reproductive or programmed cell death (apoptosis) will occur. The irradiation of surrounding healthy cells plays a minor role in the radioimmunotherapy of HIV infected T lymphocytes and HCV infected hepatocytes. This is for the reason that these cells predominantly occur in the circulation and thus, healthy cells will be subjected to irradiation only for short periods.

### DESCRIPTION OF PREFERRED EMBODIMENTS

### Preparation of Radioimmunoconjugates

1. Radioimmunopharmacons on the Basis of Monoclonal Antibodies.

The development of virus specific monoclonal antibodies and the check of their cross reactivities are bases of virological researches (Bergter 1990).

Identification and purification of cell membrane bound viral proteins of peptides belong to the state of the art (Eckert and Kartenbeck 197). Likewise, the methods to prepare murine, humanized, or human monoclonal antibodies and the preparation of antigen binding mAb fragments are known (Peters and Baumgarten 1990, Lidell and Weeks 1996). The identification and isolation of viral and virus induced antigens from the plasma membrane of effected cells and the generation of monoclonal antibody are depicted in example 1.

### EXAMPLE 1

First, it will be clarified by immunocytological and electron microscopical basic examinations whether viral proteins are principally integrated into the cell membrane of infected cells, which are susceptible to a radio immunotherapy. For this purpose, the binding of virus specific antibodies to infected cells is detected by incubation with labeled murine monoclonal antibodies (Payne et al. 1990, Stirling 1990, Kaito et al. 1994, Sabri et al. 1997) by means of fluorescence or electron microscopy.

If such antigens can be detected in the cytoplasm membrane it must be clarified in a second step, which antigens have been detected. Therefore, virus infected cells are lysed and the membrane proteins separated by gel electrophoresis. Blotted to nitrocellulose the membrane proteins will be subsequently analyzed with antiserum or murine monoclonal antibodies.

For the immunization and generation of monoclonal antibodies purification of the viral or virus induced antigens integrated into the cell membrane that have been found is required. For this purpose, viruses are cultivated in suitable cell culture systems, for example, HIV in H9-cells, MT-4 cells, MOLT-4 cells, HUT-78 cells etc. (Bergter 1990) and HCV in DAUDI-cells (Nakajima et al. 1996) and isolated from the cell culture supernatant. First, the cells and the cell debris are spun down, second the virus is sedimented from the purified supernatant by centrifugation with 27.000×g. Third the virus pellet is resuspended in sample buffer and separated by gel electrophoresis. Finally, the antigen fraction corresponding to the antigen searched for on the plasma membrane is isolated. This antigen is used for the immunization of mice and for the preparation of monoclonal antibodies.

The monoclonal antibodies thus produced are preferably checked for cross reactivities with different virus isolates, which check occurs by means of ELISA, immunoblot, or RIPA. The suitable monoclonal antibody ideally possesses a broad cross reactivity and thereby comprises the entire quasi-species of an infected patient.

If no monoclonal antibody recognizing all virus isolates is isolated, isolate specific monoclonal antibodies are prepared which antibodies will subsequently be specifically employed for the therapy of the isolate detected from case to case.

2. Preparation of Radioimmunopharmacons Based on Host Receptor Molecules.

As has been described above as well, in the meantime cellular receptor molecules have been identified for quite a number of viruses, which receptor molecules mediate the adsorption of the viruses to the surface of the host cell. The methods to identify and generate such receptor molecules belong to the state of the art. The procedure will be described in example 2. The receptor for HIV-1 and HIV-2 is the CD4 receptor, whereas HCV probably binds to the LDL receptor (Seipp et al. 1997).

### EXAMPLE 2

The respective virus (e.g. HCV) is separated by gel electrophoresis such that the (HC) viral antigens may be isolated. Binding studies will be carried out on tissue samples (from the liver) and cell culture systems and the cellular receptor responsible for the adsorption of the virus that has been searched for is identified (Dorig et al. 1993, Treichel et al. 1997). This receptor is isolated with established methods (Suzuki et al. 1983) and the binding epitope analyzed in detail by analyzing the binding of the antigen to distinct cleavage products. The receptor molecule is finally cloned (Bergelson et al. 1998) and thus further processed on a large scale for the conjugation with the radioisotope and the therapeutic use according to the present invention.

Conjugation of Suitable Radionuclides.

The monoclonal antibodies or host receptor molecules are conjugated with a radio nuclide (alpha or beta emitter). The conjugation of radio nuclides to proteins is sufficiently described and belongs to the prior art (review in: Eckert and Kartenbeck 1996). The procedure will be depicted exemplarily in example 3 although different methods (e.g. Zalutzky et al. 1989) may be more or less suitable for distinct conjugate constructs such that the conjugation methods have to be individually adapted and optimized.

### EXAMPLE 3

Suitable monoclonal antibodies or receptor molecules for therapeutic purposes are radioactively conjugated with the chloramine T method (Hunter and Greenwood 1962) following the recipe of Eckert and Kartenbeck 1997. A radioisotope is transiently oxidized by hypochlorite which is released by chloramine T (N-chloro-p-toluene-4-sulfonamide, Na salt) in aqueous medium. Then the strongly electrophilic radioisotope in this state preferably binds to the benzene rings of the aromatic amino acids contained in the protein. To treat the monoclonal antibodies and receptor molecules, respectively, gently the reaction is terminated after a short incubation by an excess of bisulfite, whereby both the residual chloramine T and oxidized but still unbound radioisotopes are reduced and thus deactivated. Finally, the mAb-radioisotope conjugate is isolated by gel electrophoresis and further processed with distinct auxiliaries for its use, preferably for its intravenous use. Further methods are likewise available (Harrison and Royle, 1984; Zalutsky et al. 1989). According to any of these methods, for instance, the conjugates a7) may be prepared.

### Preclinical Investigations

In vitro investigations show whether and with which affinity the radio immunoconjugate bind via the antigen binding site of the monoclonal antibodies or host receptor molecules, to the corresponding epitopes of viral proteins integrated into the cell membrane of virus infected cells, and whether the cells are damaged by the radioisotope mediated radiation. These investigations are performed in suitable cell culture systems.

In the following steps preclinical in vivo investigations relating to the effectiveness of the radioimmunotherapy may be performed on nude mice and other animal models before the clinical application is tested in infected patients. In case of the HCV treatment HCV infected chimpanzees (Tabor et al. 1978, Walker et al. 1997) are an example for a suitable test system.

### Application to Patients: Therapeutical Preconditions

In different phase I studies (dose escalation studies) the maximally tolerable dose (MTD) in regard of side effects, pharmacokinetics and immunogenicity are to be determined. By the subsequent clinical investigations (phase II and III studies) the effect of distinct radioimmunopharmacons shall finally be checked with small groups of patients suffering from progressive disease and with randomized groups of patients (Fiebig 1995).

The short half life of the radionuclide (be they α- or β-emitters) is responsible for the requirement that the radioimmunoconjugate is prepared in the vicinity of a center and its fast transport to the therapist, respectively. The short half life is relevant in so far as longer half-lives would expose the patient to a radiation dose too big and too long. Prerequisite for the therapy of patients suffering from HIV, viral hepatitis and, optionally, other viral infections by means of radionuclides having short half lives is thus the installation of specialized interdisciplinary centers, in which not only the professional therapy of the infected patients but also an application in time of the radioimmunoconjugate under aspects of anti-radiation precautions is secured.

Prerequisite for a successful therapy may also be the pretherapeutic decrease of the virus load which can be accomplished by a previous treatment of the patient with antiviral or anti-retroviral agents. Thereby, a higher dose of the radioimmunopharmacon is brought to the virus replicating cells, and this brings about an improved therapeutic effect. Otherwise, it would be conceivable that the radioimmunopharmacon is caught by free virus particles and the cytotoxic effect is decreased. In case of an HCV infection a prior treatment with IFN-α or ribavirin may occur. This may be done as a mono- or a combination therapy.

Prior to the application of a preparation based on ¹³¹I diagnostics and blockage of the thyroid gland following the well known specimen must additionally occur.

For the radioimmunotherapy a stationary accommodation for several days is required in order to shield the patient from the surrounding until the radiation eases off. The radioimmunopharmacon is administered periphero- or centrovenously as bolus, short infusion, or permanent therapy for several days in a dosage of 25-300 mCi, preferably 50-300, more preferred 100-200 mCi. The dosage is administered once or in cycles by repeating the administration in intervals of several weeks. Optionally, in case of an existing hypersensitiveness against the monoclonal antibody or against the receptor molecule, a previous treatment with a glucocorticoid, with an anti-histamine and/or with an H₂-antagonist is required immediately prior to the administration of the preparation (Lorenz 1994).

A particularly preferred embodiment of the invention is the use of humanized and human monoclonal antibodies, whereby the immunogenicity of murine mAb conjugates can be circumvented. Such immunogenicity may proof to make sense, on the other hand, if it is desired to additionally sensibilize the immune system against the virus infection.

A further preferred embodiment is directed to the use of fragments of monoclonal antibodies or cell receptors as immunologically effective component of the radioimmunoconjugates since the smaller molecule size may result in an improved capacity to pass through tissue and to pass the blood-brain-barrier.

The radioimmunotherapy of viral infections have a tremendous preventive significance, in particular in cases in which a preventive elimination of virus is to avoid and to combat, respectively, oncological diseases as they are known, for example as a consequence of HIV, HTLV-1, HTLV-2, HHV8, EBV, HCV and HBV infections.

Although beta and alpha emitters have entered into the radioimmunotherapy of malignant diseases, the above described therapy is, quite in contrast thereto, not only an entirely new indication or radioimmunopharmacons generally, but a basically different therapeutical approach and claim for highly specifically constructed antiviral preparations with very exactly defined areas of application. Whereas monoclonal antibodies against tumor specific proteins of the cell are used in the radioimmunotherapy of malignant diseases, for the above described radioimmunoconjugates, monoclonal antibodies, their fragments, or other proteins and peptides with the therapeutical object specifically against viral (i.e., not of the cell) and virus induced (not cell type specific) proteins.

### LITERATURE

1. Alter H J, Purcell R H, Holland P V et al.: Transmissible agent in non-A, non-B hepatitis. Lancet 1978; 1: 459-463.

2. Berg T and Hopf U: Interferontherapie der HCV-lnfektion: In: Häussinger D, Niederau C: Hepatitis C. Blackwell Wissenschafts-Verlag Berlin 1997: 229-267.

3. Bergelson J M, Krithivas A, Celi L, et al.: The murine CAR homolog is a receptor for coxsackie B viruses and adenoviruses. J Virol 1998; 72 (1): 415-419.

4. Bergter W: Herstellung zehn monoklonaler Antikörper gegen das Affen-Immundefizienzvirus SIVagmTYO-7 aus der Familie der Retroviren. Dissertation, Hannover 1990.

5. Blumenthal R D, Sharkey R M, Haywood L, et al.: Targeted therapy of athymic mice bearing GW-39 human colonic cancer micrometastases with ¹³¹I-labelled monoclonal antibodies. Cancer Res 1992; 52: 6036-6044.

6. Blumenthal R D, Sharkey R M, Natale A M, et al.: Comparison of equitoxic radioimmunotherapy and chemotherapy in the treatment of human colonic cancer xenografts. Cancer Res 1994; 54: 142-151.

7. Böker, K H W and Manns M P: Was bringt die Interferontherapie bei der chronischen Hepatitis. Internist 1997; 38: 1191-1203.

8. Boulan E R, Pendergast M: Polarized distribution of viral envelope proteins in the plasma membrane of infected epithelial cells. Cell 1980; 20 (1): 45-54.

9. Brodt H R, Helm E B, Kamps B S: AIDS 1997. Diagnostik und Therapie. Steinhauser Verlag, Wuppertal 1997: 81-117.

10. Byrn R A, Sekigawa I, Chamow S M, et al.: Characterization of in vitro inhibition of human immunodeficiency virus by purified recombinant CD4. J. Virol. 1989; 63(10): 4370-4375.

11. Choo Q L, Kuo G, Weiner A J, et al.: Isolation of a cDNA clone derived from a blood-borne non-A, non-B viral hepatitis genome. Science 1989; 244: 359-362.

12. Clapham P R, Weber J N, Whitby D, et al.: Soluble C D4 blocks the infectivity of diverse strains of HIV and SIV for T cells and monocytes but not for brain and muscle cells. Nature 1989; 337(6205): 368-370.

13. Ciampor F, Sidorenko E V, Taikova N V: Ultrastructural localization by immunoperoxidase techniques of influenza virus antigens in abortive infection of L cells. Acta Virol 1981; 25 (6): 381-389.

14. Conill C, Alsina M, Verger E, et al.: Radiation therapy in AIDS-related cutaneous Kaposi's sarcoma. Dermatology 1997;195 (1): 40-42.

15. Chu C M, Liaw Y F: Natural history of chronic hepatitis B virus infection: an immunopathological study. J Gastroenterol Hepatol 1997; 12 (9-10): 218-222.

16. Dalgleish A G, Beverly P C, Clapham P R et al.: The CD4 (T4) antigen is an essential component of the receptor for the AIDS retrovirus. Nature 1984; 312 (5996): 763-767.

17. Darai G. et al.: Lexikon der Infektionskrankheiten des Menschen. Springer, Heidelberg 1997

18. Deen K C, McDougal JS, Inacker R et al.: A soluble form of CD4 (T4) protein inhibits AIDS virus infection. Nature 1988; 331(6151): 82-84.

19. Dennin R H, Beyer A: Application of scanning electron microscopy (SEM) and microbead techniques to study the localization of p24 and p18 antigens of HIV-1 on the surface of HIV-1-infected H9-lymphocytes. J Microsc 1991;164 (Pt 1): 53-60.

20. Dorig R E, Marcil A, Chopra A, et al.: The human CD46 molecule is a receptor for measles virus (Edmonstron strain). Cell 1993; 75 (2): 295-305.

21. Desportes I, Bonnet D, Nicol I, et al.: Expression of HIV antigens at the surface of infected T4 cells: immunoelectron microscopic evidence of an immunogenic phase prior to the viral release. AIDS Res Hum Retroviruses 1989; 5 (1): 107-114.

22. Dudhane A, Wang Z Q, Orlikowsky T, et al.: AIDS patient monocytes target CD4 T cells for cellular conjugate formation and deletion through the membrane expression of HIV-1 envelope molecules. AIDS Res Hum Retroviruses 1996; 12 (10): 893-899.

23. Eckert W A and Kartenberg J: Proteine: Standardmethoden der Molekular- und Zell-biologie. Springer, Heidelberg 1997: 6-12, 237-240.

24. Erickson J W and Burt S K: Structural mechanisms of HIV drug resistance. Annu Rev Pharmacol Toxicol 1996; 36: 545-571.

25. Farci P, Shimoda A, Wong D, et al.: Prevention of hepatitis C virus infection in chimpanzees by hyperimmune serum against the hypervariable region 1 of the envelope 2 protein. Proc Natl Acad Sci USA 1996; 93 (26): 15394-15399.

26. Feremans W W, Huygen K, Menu R, et al.: Fifty cases of human immunodeficiency virus (HIV) infection: immunoultrastructural study of circulating lymphocytes. J Clin Pathol 1988; 41 (1): 62-71.

27. Fetzer I. C., Bergter W, Ramadori G: Kombinationstherapie mit INF-a2a und Ribavirin nach erfolgloser IFN-α2a-Monotherapie bei Patienten mit chronischer HCV-Infektion. Göttinger Erfahrung. Z Gastroenterol 1997; 35: 419-433.

28. Fiebig H H: Durchfuhrung von klinischen Studien. In Zeller W J, Zur Hausen H (Hrsg): Onkologie, Grundlagen, Diagnostik, Therapie, Entwicklungen. Ecomed, Landsberg/Lech 1995; IV-2: 1-5.

29. Fingeroth J D; Weis J J; Tedder T F et al.: Epstein-Barr virus receptor of human B lymphocytes is the C3d receptor CR2. Proc Natl Acad Sci USA 1984; 81 (14): 4510-4514.

30. Follea G, Herve P, Andreu G, et al.: Viral attenuation of labile blood products. Transfus Clin Biol 1996; 3 (2): 113-123.

31. Fultz P N, Schwiebert R S, Su L, et al.: Effects of total lymphoid irradiation on SIV-infected macaques. AIDS Res Hum Retroviruses 1995; 11(12): 1517-1527.

32. Gaubin M, Autiero M, Houlgatte R, et al.: Molecular basis of T lymphocyte CD4 antigen functions. Eur J Clin Chem Clin Biochem 1996;34(9): 723-728.

33. Gerber M A, Sells M A, Chen M L, et al.: Morphologic, immunohistochemical, and ultrastructural studies of the production of hepatitis B virus in vitro. Lab Invest 1988; 59 (2): 173-180.

34. Glick B R and Pasternak J J: Molekulare Biotechnologie. Spektrum Akademischer Verlag Heidelberg 1995: 91-164.

35. Harrison A, Royle L: Preparation of a 211At-IgG conjugate which is stable in vivo. Int J Appl Radiot Isot 1984; 35(11): 1005-1008.

36. Hughey P G, Compans R W, Zebedee S L, et al.: Expression of the influenza A virus M2 protein is restricted to apical surfaces of polarized epithelial cells. J Virol 1992; 66 (9): 5542-5552.

37. Hunter W M, Greenwood F C: Preparation of iodine-131 labelled human growth hormone of high specific activity. Nature 1962; 194: 495-496.

38. Imrie A, Beveridge A, Genn W: Transmission of human immunodeficiency virus type 1 resistant to nevirapine and zidovudine. Sydney Primary HIV Infection Study Group. J Infect Dis 1997;175(6): 1502-1506.

39. Ikuta K, Morita C, Miyake S, et al.: Expression of human immunodeficiency virus type 1 (HIV-1) gag antigens on the surface of a cell line persistently infected with HIV-1 that highly expresses HIV-1 antigens. Virology 1989; 170 (2): 408-417.

40. Jones D H, Sakamoto K, Vorce R L, Howard B J: DANN mutagenesis and recombination. Nature 1990; 344: 793-794.

41. Kaito M, Watanabe S, Tsukiyama-Kohara K, et al.: Hepatitis C virus particle detected by immunoelectron microscopic study. J Gen Virol 1994; 75 (Pt 7): 1755-1760.

42. Kaminski M S, Zasadny K R, Francis I R, et al.: Radioimmunotherapy of B-cell lymphoma with [¹³¹J]anti-B1 (anti-CD20) antibody. N Engl J Med 1993; 329: 459-465.

43. Kaplan G, Totsuka A, Thompson P, et al.: Identification of a surface glycoprotein on African green monkey kidney cells as a receptor for hepatitis A virus. EMBO J 1996; 15 (16): 4282-4296.

44. Kauffmann G, Moser E, Sauer R: Radiologie. Grundlagen der Radiodiagnostik, Radiotherapie und Nuklearmedizin. Urban und Schwarzenberg München 1996.

45. Kilby J M et al., Potent suppression of HIV-1 replication in humans by T-20, a peptide inhibitor of gp41-mediated virus entry. Nat. Med. 4(11): 1302-1307 (1998).

46. Kohama M T, Cardenas J M, Seto J T: Immunoelectron microscopic study of the detection of the glycoproteins of influenza and Sendai viruses in infected cells by the immunoperoxidase method. J Virol Methods 1981;3 (5): 293-301.

47. Kozak R W, Atcher R W, Gansow O A, et al.: Bismuth-212-labelled anti-Tac monoclonal antibody: alpha-particle-emitting radionuclides as modalities for radioimmunotherapy. Proc Natl Acad Sci USA 1986; 83(2): 474-478.

48. Krain J, Dieckmann K P: Treatment of tsticular seminoma in patients with HIV infection. Report of two cases. Eur Urol 1994; 26(2): 184-186.

49. Lentz T L, Benson R J, Klimowicz D, et al.: Binding of rabies virus to purified Torpedo acetylcholine receptor. Brain Res. 1986; 387 (3): 211-219.

50. Leon-Monzon M E, IIIa I, Dalakas M C: Expression of poliovirus receptor in human spinal cord and muscle. Ann N Y Acad Sci 1995; 753: 48-57.

51. Liddell E und Weeks I: Antikörper-Techniken. Spektrum Akademischer Verlag, Heidelberg 1996.

52. Liebowitz D, Wang D, Kieff E: Orientation and patching of the latent infection membrane protein encoded by Epstein-Barr virus. J Virol 1986; 58 (1): 233-237.

53. Lorenz R: Bildgebende Verfahren. In: Classen M, Diehl V, Kochsiek K: Innere Medizin. Urban & Schwarzenbeck München 1994 (3. Auflage): 39-63.

54. Lotz R, Rosler H P, Zapf S, et al.: Radiotherapy in HIV positive patients. Fortschr Med 1990; 108(2): 35-36,39.

55. Macklis R M, Kinsey B M, Kassis A I, et al.: Radioimmuntotherapy with alpha-particle-emitting immunoconjugates. Science 1988; 240(4855): 1024-1026.

56. Maier K P: Pathologie der Hepatitis C. In: Häussinger D, Niederau C: Hepatitis C. Blackwell Wissenschafts-Verlag Berlin 1997: 149-163.

57. Manns M P und Rambusch E G: Extrahepatische Manifestationen und Autoimmunitat bei HCV-Infektion. In: Ha ussinger D, Niederau C: Hepatitis C. Blackwell Wissenschafts-Verlag Berlin 1997: 165-182.

58. Martin C E and Scheinbach S: Expression of proteins encoded by foreign genes in Saccharomyces cerevisiae. Biotech Adv 1989; 7: 155-185.

59. Moser E: Nuklearmedizin. In Kauffmann G, Moser E und Sauer R: Radiologie. Urban und Schwarzenberg, München 1996: 253-283.

60. Muster T, Steindl F, Purtscher M, et al.: A conserved neutralizing epitope on gp41 of human immunodeficiency virus type 1. J Virol 1993; 67(11): 6642-6647.

61. Nakajima A, Hijikata M, Yoshikura H, et al.: Charakterization of longterm cultures of hepatitis C virus. J Virol 1996; 70: 3325-3329.

62. Ng M I, Choo Wk, Ho Y I: Detection of flavivirus antigens in purified infected Vero cell plasma membranes. J Virol Methods 1992; 39 (1-2): 125-138.

63. Nishimura H, Sugawara K, Kitame F, et al.: Attachment of influenza C virus to human erythrocytes. J Gen Virol 1988; 69 (Pt 10): 2545-2553.

64. Payne H R; Storz J; Henk W G: Bovine coronavirus antigen in the host cell plasmalemma. Exp Mol Pathol 1990; 53 (2): 152-159.

65. Peters J H and Baumgarten H: Monoklonale Antikorper. Herstellung und Charakterisierung. Springer, Berlin 1990 (2. Auflage).

66. Petry et al.: Hepatozellulares Karzinom bei HCV-lnfektion. In: Häussinger D, Niederau C: Hepatitis C. Blackwell Wissenschafts-Verlag Berlin 1997: 183-219.

67. Plettenberg A, Janik I, Kolb H, et al.: Local therapy measures in HIV-associated Kaposi's sarcoma with special reference to fractionated radiotherapy. Strahlenther Onkol 1991; 167(4):208-213.

68. Pozzetto B, Bourlet T, Grattard F et al.: Structure, genomic organization, replication and variability of hepatitis C virus. Nephrol Dial Transplant 1996; 11 (Suppl 4): 2-5.

69. Press O W, Eary J F, Appelbaum F R, et al.: Radiolabelled-antibody therapy of B-cell lymphoma with autologous bone marrow support. N Engl J Med 1993; 329:1219-1224.

70. Press O W, Eary J F, Appelbaum F R, et al.: Phase II trial of ¹³¹J-B1 (anti-CD20) antibody therapy with autologous stem cell transplantation for relapsed B-cell lymphoma. Lancet 1995; 346: 336-340.

71. Press O W, Eary J F, Appelbaum F R, et al.: Treatment of relapsed B-cell lymphomas with high dose radioimmunotherapy and bone marrow transplantation. In Goldenberg D M (ed.): Cancer Therapy with Radiolabelled Antibodies. Boca Raton, CRC Press 1995: 229-237.

72. Purcell R: The hepatitis C virus: overview. Hepatology 1997;26 (3 Suppl 1): 11S-14S.

73. Reichard 0, Yun Z B, Sonnerborg A, et al.: Hepatitis C viral RNA titers in serum prior to, during, and after oral treatment with ribavirin for chronic hepatitis C. J Med Virol 1993; 41: 99-102.

74. Reiners C: Zum Krebs- und genetischen Risiko nach Radioiodtherapie der Hyperthyreose. Der Nuklearmediziner 1997; 5 (20): 331-334.

75. Revilla-Monsalve C, Hernandez-Jauregui P, Silva-Lemoine E: Immunoperoxidase cell surface localization of rabies virus antigen in tissue cultures at a low viral multiplicity. Arch Invest Med (Mex) 1985; 16 (1): 11-17.

76. Rosa D, Campagnoli S, Moretto C, et al.: A quantitative test to estimate neutralizing antibodies to the hepatitis C virus: cytofluorometric assessment of envelope glycoprotein 2 binding to target cells. Proc Natl Acad Sci USA 1996; 93 (5): 1759-1763.

77. Rusche J R, Lynn D L, Robert-Guroff M, et al.: Humoral immune response to the entire human immunodeficiency virus envelope glycoprotein made in insect cells. Proc Natl Acad Sci USA 1987; 84 (19): 6924-6928.

78. Sabri S, Richelme F, Pierres A et al.: Interest of image processing in cell biology and immunology. J Immunol Methods 1997; 208 (1): 1-27.

79. Saito T, Kamimura T, Ishibashi M, et al.: Electron microscopic study of hepatitis B virus-associated antigens on the infected liver cell membrane in relation to analysis of immune target antigens in chronic hepatitis B. Gastroenterol Jpn 1992; 27 (6): 734-744.

80. Santolini E, Pacini L, Fipaldini C, et al.: The NS2 protein of hepatitis C virus is a transmembrane polypeptide. J Virol 1995; 69 (12): 7461-7471.

81. Saran F, Adamietz I A, Mose S, et al.: The value of conventionally fractionated radiotherapy in the local treatment of HIV-related Kaposi's sarcoma. Strahlenther Onkol 1995; 171(10): 594-599.

82. Saran F H, Adamietz I A, Thilmann C, et al.: HIV-associated cutaneous Kaposi's sarcoma—palliative local treatment by radiotherapy. Acta Oncol 1997; 36(1): 55-58.

83. Sattentau Q J, Clapham P R, Weiss R A, et al.: The human and simian immunodeficiency viruses HIV-1, HIV-2 and SIV interact with similar epitopes on their cellular receptor, the CD4 molecule. AIDS 1988; 2(2): 101-105.

84. Sauer R: Strahlenbiologie. In: Kauffmann G, Moser E und Sauer R: Radiologie. Urban und Schwarzenberg, München 1996: 31-80.

85. Sauer R: Strahlentherapie und Onkologie für MTA-R. Urban und Schwarzenberg, München 1998.

86. Scheidegger C, Heinrich B, Popescu M, et al.: HIV associated lymphomas. Dtsch Med Wochenschr 1991; 116(30):1129-1135.

87. Schlehofer J R, Hampl H, Habermehl K O: Differences in the morphology of herpes simplex virus infected cells: I. Comparative scanning and transmission electron microscopic studies on HSV-1 infected HEp-2 and chick embryo fibroblast cells. J Gen Virol 1979; 44 (2): 433-442.

88. Schvarcz R, Yun Z B, Sonnerborg A et al.: Combined treatment with interferon alpha-2b and ribavirin for chronic hepatitis C in patients with a previous non-response or non-sustained response to interferon alone. J Med Virol 1995; 46 (1): 43-47.

89. Seipp S, Mueller H M, Pfaff E, et al.: Establishment of persistent hepatitis C virus infection and replication in vitro. J Gen Virol. 1997; 78 (Pt10): 2467-2476.

90. Selby M J, Choo Q L, Berger K, et al.: Expression, identification and subcellular localization of the proteins encoded by the hepatitis C viral genome. J Gen Virol 1993; 74 (Pt 6): 1103-1113.

91. Shafren D R, Williams D T, Barry R D: A decay-accelerating factor-binding strain of coxsackievirus B3 requires the coxsackievirus-adenovirus receptor protein to mediate lytic infection of rhabdomyosarcoma cells. J Virol 1997; 71 (12): 9844-9848.

92. Shimotohno K, Tanji Y, Hirowatari Y et al.: Processing of the hepatitis C virus precurser protein. J Hepatol 1995; 22 (Suppl.1): 87-92.

93. Stirling J W: Immuno- and affinity probes for electron microscopy: a review of labeling and preparation techniques. J Histochem Cytochem 1990; 38 (2): 145-157.

94. Su S B et al.: T20/DP178, an ectodomain peptide of human immunodeficiency virus type 1 gp41, is an activator of human phagocyte N-formyl peptide receptor. Blood 93(11):3885-3892 (1999)

95. Suzuki Y, Suzuki T, Matsumoto M: Isolation and characterization of receptor sialoglycoprotein for hemagglutinating virus of Japan (Sendai virus) from bovine erythrocyte membrane. J Biochem (Tokyo) 1983; 93 (6): 1621-1633.

96. Swift P S: The role of radiation therapy in the management of HIV-related Kaposi's sarcoma. Hematol Oncol Clin North Am 1996; 10(5): 1069-1080.

97. Tabor E, Gerety R J, Drucker J A: Transmission of non-A, non-B hepatitis from man to chimpanzee. Lancet 1978; 1: 463-466.

98. Timar J, Nagy K, Lapis K: Morphologic and immunoelectronmicroscopic identification of human T-cell lymphotropic virus type III (HTLV-III). Histol Histopathol 1986;1 (1): 43-47.

99. Treichel U, Meyer zum Büschenfelde, Dienes H-P et al.: Receptor-mediated entry of hepatitis B virus particles into liver cells. Arch Virol 1997; 142: 493-498.

100. Turkington T G, Zalutzky M R, Jaszczak R J, Garg P, Vaidynathan G, Coleman R E. Measuring astatine-211 distributions with SPECT. Phys Med Biol 1993; 38:1121-1130.

101. Vento S, Concia E, Ferraro T: Lack of sustained efficiacy of interferon in patients with chronic hepatitis C. N Engl J Med 1996; 334: 1479.

102. Walker C M: Comparative features of hepatitis C virus infection in humans and chimpanzees. Springer Semin Immunopathol 1997;19 (1): 85-98.

103. Watanabe M, Reimann K A, DeLong P A, et al.: Effect of recombinant soluble CD4 in rhesus monkeys infected with simian immunodeficiency virus of macaques. Nature 1989; 337(6204): 267-272.

104. Westaway E G, Goodman M R: Variation in distribution of the three flavivirus-specific glycoproteins detected by immunofluorescence in infected Vero cells. Arch Virol 1987; 94 (3-4): 215-228.

105. Whitbeck J C, Peng C, Lou H, et al.: Glycoprotein D of herpes simplex virus (HSV) binds directly to HVEM, a member of the tumor necrosis factor receptor superfamily and a mediator of HSV entry. J Virol 1997; 71 (8): 6083-6093.

106. Wilcox G and Studnicka G M: Expression of foreign proteins in microorganisms. Biotech Appl. Biochem 1988; 10: 500-509.

107. Xu Y, Conway B, Montaner J S, et al.: Effect of low-dose gamma radiation of HIV replication in human peripheral blood mononuclear cells. Photochem Photobiol 1996; 64(2): 238-241.

108. Zalutzky M R and Bigner D D: Radioimmunotherapy with a-particle emitting radioimmunoconjugates. Acta Oncol 1996; 35(3): 373-379.

109. Zalutzky M R, Garg P K, Friedmann H S et al.: Labeling monoclonal antibodies and F(ab′)₂ fragments with the alpha-particle emitting nuclide astatine-211: preservation of immunoreactivity and in vivo localizing capacity. Proc Natl Acad Sci USA 1989; 86(18): 7149-7153.

110. Zilpert A und Roggendorf M: Molekularbiologie des Hepatitis-C-Virus. In: Häussinger D, Niederau C: Hepatitis C. Blackwell Wissenschafts-Verlag Berlin 1997: 13-51.

111. Zweit J: Radionuclides and carrier molecules for therapy. Phys Med Biol 1996; 41: 1905-1914.

What is claimed is:

1. A method of treating viral infections and tumors induced thereby, the method comprising the steps of:
combining an immunologically effective component and a radioactive component to a radioimmunoconjugate (RIC), wherein the immunologically effective component is:
(a) a monoclonal antibody and its antigen binding fragment, respectively, against a viral or virus induced antigen expressed on the plasma membrane of virus infected cells,
and wherein the radioactive component is
(b) an alpha emitter or a beta emitter,
administering the radioimmunoconjugate (RIC) subsequent to or during an anti-retroviral therapy such as the standard triple therapy, and/or, for a HBV, HCV or HDV infection, subsequent to or during an IFN-α mono- or combination therapy with ribavirin, with the proviso that the RIC is not a therapeutical agent for the treatment of HIV infections and of tumors induced thereby, if the monoclonal antibody or its fragment comprises a beta emitter as a radioactive component.

2. The method of claim 1, wherein the viral infection is a HIV, HBV, HCV, HDV, HTLV, CMV, EBV, or HHV8 infection.

3. The method of claim 2, wherein the infection is an infection with HIV-1, HIV-2, or HIV-3, or an infection with HTLV-1 or HTLV-2.

4. The method of claim 1, wherein, in the step of administering, the radioimmunoconjugate is given intravenously several times for a total body dosage of 25-300 mCi under in-patient radiation shielding for one day up to several days.

5. The method of claim 1, wherein the α- and β-emitter, respectively, has a short half-life of 10 days and less.

6. The method of claim 5, wherein the α-emitter is or contains at least one of bismuth-213, astatine-211, radium-223, actinium-225, and wherein the β-emitter is or contains at least one of ¹³¹I, ⁸⁹Sr, ¹⁷⁷Lu, ¹⁸⁶Lu, ¹⁸⁶Re, ¹⁸⁸Re, ¹⁰⁵Rh, ⁴⁷Sc, ¹⁵³Sm, and ¹⁴⁹Tb.

7. The method according to claim 1, wherein the step of administering is performed:
subsequent to or during an anti-retroviral therapy such as the standard triple therapy in the case of a HIV infection;
subsequent to or during an IFN-α mono or combination therapy with ribavirin in the case of a HBV, HCV or HDV infection;
before, during or subsequent to a surgical procedure, in particular a transplantation of a cirrhotically modified liver due to a viral hepatitis or a resection of a hepatocellular carcinoma induced by a viral hepatitis;
under the protection of a stem cell transplantation;
before, during or subsequent to an administration of an immunological component, as defined in claim 1, without a radioactive component.
